(19) United States
(12) Patent Application Publication
(10) Pub. No.: US 2026/0090775 A1
(43) Pub. Date: Apr. 2, 2026

(54) DATA PROCESSING DEVICE, DATA PROCESSING METHOD, AND DATA PROCESSING PROGRAM

(71) Applicant: FUJIFILM Corporation, Tokyo (JP)

(72) Inventor: Fuyuhiko TERAMOTO, Tokyo (JP)

(73) Assignee: FUJIFILM Corporation, Tokyo (JP)

(21) Appl. No.: 19/325,572

(22) Filed: Sep. 11, 2025

(30) Foreign Application Priority Data
Sep. 27, 2024 (JP) 2024-169514

(51) Int. Cl. A61B 6/42; A61B 6/00; A61B 6/58

(52) U.S. Cl. CPC A61B 6/4241; A61B 6/482; A61B 6/5258; A61B 6/585

(57) ABSTRACT

A processor is configured to acquire projection data that is obtained by detecting radiation emitted from a radiation source and transmitted through a subject via a photon-counting detector consisting of a plurality of detection elements and that corresponds to the number of photons of the radiation in each of a plurality of energy bins of the radiation, derive first correction data for correcting projection data of at least one target energy bin that is a correction target among the plurality of energy bins based on the projection data in each of the plurality of energy bins, and derive first corrected projection data by correcting, in the projection data of the target energy bin, a projection value that is equal to or less than a first threshold value in the first correction data using the first correction data.

### CROSS-REFERENCE TO RELATED APPLICATION

The present application claims priority from Japanese Patent Application No. 2024-169514, filed on Sep. 27, 2024, the entire disclosure of which is incorporated herein by reference.

### BACKGROUND

### Technical Field

The present disclosure relates to a data processing device, a data processing method, and a data processing program.

### Related Art

In recent years, a photon-counting computed tomography (PCCT) apparatus, which is a radiography apparatus provided with a photon-counting detector (PCD), has been known. Unlike a charge-integrating detector employed in a computed tomography (CT) apparatus in the related art, the PCD can acquire projection data obtained by measuring the number of incident photons of radiation for each of a plurality of energy bins. Therefore, a larger amount of information can be obtained than in the CT apparatus in the related art.

In the PCD, pile-up in which the apparent energy of the photon appears to be high or a count loss that causes the measured count value to be smaller than an ideal value occurs, and there is a concern that the measured count value deviates from a true count value. For this reason, various methods for correcting the projection data have been proposed. For example, JP2019-181160A proposes a method of acquiring projection data including a count corresponding to energy bins and estimating a spectrum of the count affected by pile-up, to correct the projection data.

Since the pile-up and the count loss described above occur significantly in a case where a radiation dose is large, the influence of the pile-up and the count loss is likely to appear in a region in which the radiation dose is large, that is, in which a projection value in the projection data with a small subject is low.

### SUMMARY OF THE INVENTION

The present disclosure has been made in view of the circumstances described above, and an object of the present disclosure is to enable appropriately correction of an influence of pile-up or the like in projection data.

The present disclosure relates to a data processing device comprising: a processor configured to acquire projection data that is obtained by detecting radiation emitted from a radiation source and transmitted through a subject via a photon-counting detector consisting of a plurality of detection elements and that corresponds to the number of photons of the radiation in each of a plurality of energy bins of the radiation, derive first correction data for correcting projection data of at least one target energy bin that is a correction target among the plurality of energy bins based on the projection data in each of the plurality of energy bins, and derive first corrected projection data by correcting, in the projection data of the target energy bin, a projection value that is equal to or less than a first threshold value in the first correction data using the first correction data.

In the data processing device according to the present disclosure, the processor may be configured to calibrate the projection data in each of the plurality of energy bins, and derive the first correction data and the first corrected projection data using the calibrated projection data.

In the data processing device according to the present disclosure, the processor may be configured to derive the first corrected projection data by correcting, in the projection data of the target energy bin, a projection value that is equal to or greater than a second threshold value, which is less than the first threshold value, and less than the first threshold value in the first correction data to a projection value derived by an interpolation operation between the projection value of the projection data of the target energy bin and the projection value of the first correction data, and correcting a projection value that is less than the second threshold value in the first correction data to the projection value of the first correction data.

In the data processing device according to the present disclosure, the processor may be configured to set at least one of the first threshold value or the second threshold value based on at least one of energy of the target energy bin, positions of the detection elements in accordance with a shape of a compensator that compensates a dose of the radiation emitted to the subject, or a tube current of the radiation.

In the data processing device according to the present disclosure, the target energy bin may be at least one of a maximum energy bin or a minimum energy bin among the plurality of energy bins, and the processor may be configured to derive the first correction data by assigning a relatively larger weight to the projection data of energy bins other than the maximum energy bin and the minimum energy bin among the plurality of energy bins than to the projection data of the maximum energy bin and the minimum energy bin, and performing a weighted-combination of a plurality of the projection data.

In the data processing device according to the present disclosure, the processor may be configured to derive second correction data for correcting a contrast of the first corrected projection data in accordance with a size of the subject based on the projection data in each of the plurality of energy bins, and derive second corrected projection data by performing a weighted-combination of the second correction data and the first corrected projection data in accordance with the size of the subject.

In the data processing device according to the present disclosure, the processor may be configured to derive the second correction data by assigning a relatively larger weight to the projection data of an energy bin in which a variation in the projection value due to the size of the subject is relatively smaller than other energy bins among the plurality of energy bins than to the projection data of the other energy bins, and performing a weighted-combination of a plurality of the projection data.

In the data processing device according to the present disclosure, the processor may be configured to derive an indicator indicating the size of the subject based on an added value of the projection values of the second correction data.

The present disclosure relates to a data processing method executed by a computer, comprising: acquiring projection data that is obtained by detecting radiation emitted from a radiation source and transmitted through a subject via a photon-counting detector consisting of a plurality of detection elements and that corresponds to the number of photons of the radiation in each of a plurality of energy bins of the radiation; deriving first correction data for correcting projection data of at least one target energy bin that is a correction target among the plurality of energy bins based on the projection data in each of the plurality of energy bins; and deriving first corrected projection data by correcting, in the projection data of the target energy bin, a projection value that is equal to or less than a first threshold value in the first correction data using the first correction data.

The present disclosure relates to a data processing program causing a computer to execute a procedure comprising: acquiring projection data that is obtained by detecting radiation emitted from a radiation source and transmitted through a subject via a photon-counting detector consisting of a plurality of detection elements and that corresponds to the number of photons of the radiation in each of a plurality of energy bins of the radiation; deriving first correction data for correcting projection data of at least one target energy bin that is a correction target among the plurality of energy bins based on the projection data in each of the plurality of energy bins; and deriving first corrected projection data by correcting, in the projection data of the target energy bin, a projection value that is equal to or less than a first threshold value in the first correction data using the first correction data.

It should be noted that the technology of the present disclosure may be applied to a program product.

According to the present disclosure, the influence of the pile-up or the like in the projection data can be appropriately corrected.

### BRIEF DESCRIPTION OF THE DRAWINGS

FIG. 1 is a diagram showing a schematic configuration of a data processing device according to an embodiment of the present disclosure.

FIG. 2 is a diagram showing a hardware configuration of the data processing device according to the present embodiment.

FIG. 3 is a diagram showing a functional configuration of the data processing device according to the present embodiment.

FIG. 4 is a diagram showing deterioration of linearity of projection data for each of the energy bins.

FIG. 5 is a diagram showing settings of a first threshold value and a second threshold value.

FIG. 6 is a diagram showing derivation of first corrected projection data.

FIG. 7 is a diagram showing a relationship between a size of a subject and a weight.

FIG. 8 is a flowchart showing a process performed in the present embodiment.

### DETAILED DESCRIPTION

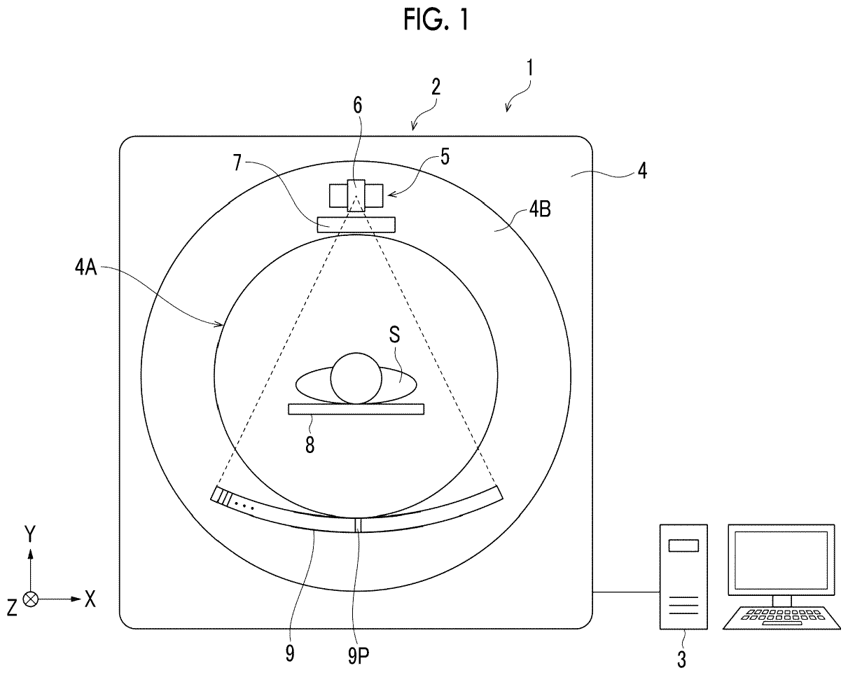

Hereinafter, an embodiment of the present disclosure will be described in detail with reference to the accompanying drawings. First, an example of a configuration of a medical imaging system comprising a control device of a data processing device according to the embodiment of the present disclosure will be described. FIG. 1 is a schematic configuration diagram of the medical imaging system comprising the data processing device according to the present embodiment.

As shown in FIG. 1, a medical imaging system 1 according to the present embodiment comprises a CT apparatus 2 and a console 3. The CT apparatus 2 comprises a gantry 4 and a table 8. It should be noted that, in the following description, a lateral direction in FIG. 1 is defined as an X axis, a longitudinal direction is defined as a Y axis, and a direction orthogonal to an XY plane is defined as a Z axis. The CT apparatus 2 is an example of a radiography apparatus.

The gantry 4 has an opening portion 4A, and a subject S as an imaging target is disposed in the opening portion 4A in a state of being placed on the table 8. The gantry 4 and the table 8 can be relatively moved in a Z axis direction.

A radiation source 5 having a radiation tube 6 and a bowtie filter 7 and a detector 9 are disposed inside the gantry 4 to face each other with the subject S interposed therebetween. The bowtie filter 7 increases a dose near the center and decreases a dose in the periphery to optimize an amount of exposure in order to suppress an amount of exposure in a peripheral portion. Radiation emitted from the radiation tube 6 is formed into a beam shape suitable for a size of the subject S by the bowtie filter 7, and the subject S is irradiated with the radiation. The bowtie filter 7 is an example of a compensator according to the present disclosure.

The detector 9 detects the radiation transmitted through the subject S, to generate projection data in accordance with the dose of the detected radiation. As an example, the detector 9 according to the present embodiment is a photon-counting detector in which a plurality of detection elements 9P that detect photon energy, which is energy of photons of the incident radiation, are disposed in an arc shape centered on a focal point of the radiation tube 6.

The photon-counting detector measures energy for each photon and outputs the projection data corresponding to the number of photons of the radiation in each of a plurality of energy bins. In the present embodiment, the detector 9 outputs, for example, the projection data for each of the four energy bins. The four energy bins can be set to, for example, less than 30 keV, 30 keV or greater and less than 50 keV, 50 keV or greater and less than 100 keV, and 100 keV or greater, but the present disclosure is not limited to this. The projection data is two-dimensional data in which a detection signal acquired in each of the plurality of detection elements 9P included in the detector 9, that is, a projection value is set as a pixel value of each pixel. The projection value also includes a signal for each of the four energy bins.

In the present embodiment, X-rays are used as an example of the radiation, but the present disclosure is not limited to this, and y-rays or the like can also be used.

The radiation tube 6 and the detector 9 are attached to a rotation plate 4B in the gantry 4 and are rotated around the subject S by a rotation drive unit (not shown). With the repetition of the radiation irradiation from the radiation tube 6 and the detection of the radiation by the detector 9 together with the rotation of the radiation tube 6 and the detector 9, the projection data is acquired in a plurality of view units in which projection angles of the radiation to the subject are different. The projection data acquired by the detector 9 is output to the console 3.

The console 3 sets the dose of the radiation emitted from the radiation tube 6, a rotation speed of the gantry 4, a relative movement speed between the gantry 4 and the table 8, and the like based on imaging conditions including a tube current and the like input by an operator, such as a technician.

The console 3 according to the present embodiment performs control related to imaging of the subject S, correction of the projection data acquired by the imaging, generation of a tomographic image from the projection data, and the like. The console 3 is an example of a data processing device according to the present disclosure.

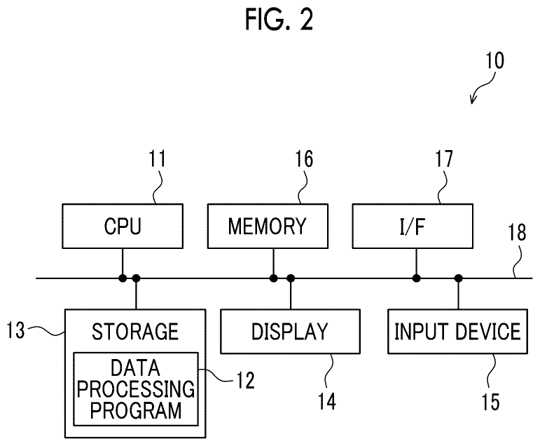

Hereinafter, the data processing device according to the present embodiment will be described. First, a hardware configuration of the data processing device according to the present embodiment included in the console 3 will be described with reference to FIG. 2. As shown in FIG. 2, the data processing device 10 included in the console 3 is a computer such as a workstation, a server computer, and a personal computer, and comprises a central processing unit (CPU) 11, a non-volatile storage 13, and a memory 16 as a transitory storage area.

In addition, the data processing device 10 comprises a display 14, an input device 15, and an interface (I/F) 17. The CPU 11, the storage 13, the display 14, the input device 15, the memory 16, and the I/F 17 are connected to a bus 18. The CPU 11 is an example of a processor according to the present disclosure.

The storage 13 is achieved by a hard disk drive (HDD), a solid state drive (SSD), a flash memory, or the like. The storage 13 as a storage medium stores a data processing program 12 installed in the data processing device 10. The CPU 11 reads out the data processing program 12 from the storage 13, loads the data processing program 12 in the memory 16, and executes the loaded data processing program 12.

The display 14 is a device that displays various screens and is, for example, a liquid-crystal display or an electro-luminescence (EL) display.

The input device 15 is used by the operator to input an instruction and various types of information related to the imaging conditions in a case of imaging the subject S, image generation, display, and the like. Examples of the input device 15 include various switches, buttons, a touch panel, a touch pen, a keyboard, and a mouse. The display 14 and the input device 15 may be integrated to form a touch panel display.

The I/F 17 communicates various types of information between the gantry 4, the rotation drive unit (not shown), the radiation source 5, and the detector 9 by wired communication or wireless communication.

The data processing program 12 is stored in a storage device of a server computer connected to a network or in a network storage in a state of being accessible from the outside, and is downloaded and installed in the computer constituting the data processing device 10 in response to a request. Alternatively, the data processing program 12 is distributed in a state of being recorded on a recording medium, such as a digital versatile disc (DVD) or a compact disc read only memory (CD-ROM), and is installed in the computer constituting the data processing device 10 from the recording medium.

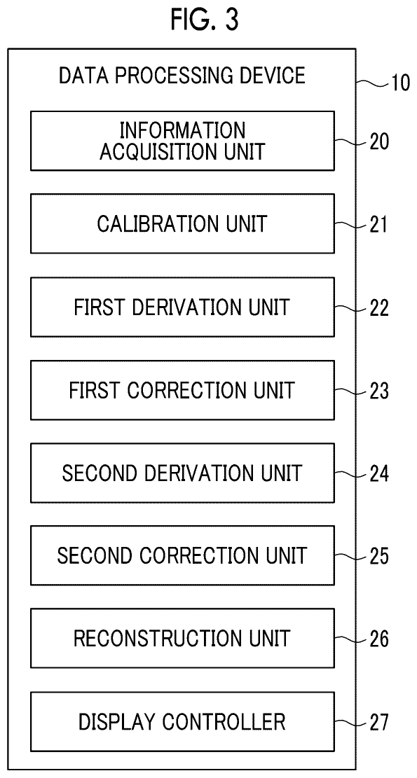

Hereinafter, a functional configuration of the data processing device according to the present embodiment will be described. FIG. 3 is a diagram showing the functional configuration of the data processing device according to the present embodiment. As shown in FIG. 3, the data processing device 10 comprises an information acquisition unit 20, a calibration unit 21, a first derivation unit 22, a first correction unit 23, a second derivation unit 24, a second correction unit 25, a reconstruction unit 26, and a display controller 27. Then, the CPU 11 executes the data processing program 12 to function as the information acquisition unit 20, the calibration unit 21, the first derivation unit 22, the first correction unit 23, the second derivation unit 24, the second correction unit 25, the reconstruction unit 26, and the display controller 27.

The information acquisition unit 20 acquires the projection data in each view, which is acquired by imaging the subject S from the CT apparatus 2. The projection data is data that corresponds to the number of photons of the radiation for each of the plurality of energy bins and that is output by the detector 9. In the present embodiment, it is assumed that first projection data P01 to fourth projection data P04 are acquired in each of the four energy bins, that is, first to fourth energy bins. The four energy bins are assumed to increase in energy from the first to the fourth.

The calibration unit 21 calibrates the first projection data P01 to the fourth projection data P04 using calibration data acquired in advance and stored in the storage 13, to derive the calibrated first projection data P1 to fourth projection data P4. Hereinafter, the calibrated first projection data P1 to fourth projection data P4 are simply referred to as the first projection data P1 to the fourth projection data P4.

The calibration data is acquired in advance by performing phantom calibration, and then stored in the storage 13. The phantom calibration is a process of acquiring the calibration data by imaging, in the CT apparatus 2, a phantom having a known density and a known transmission length, for example, a cylindrical shape, in order to calibrate the density and the transmission length of the subject S obtained by the imaging. The calibration data is acquired for each of the energy bins. The phantom calibration is executed at the time of shipment of the CT apparatus 2, at the time of regular inspection, or the like.

Here, the first to fourth projection data P1 to P4 acquired by the detector 9 may deviate from a true value in the projection value (that is, a signal value corresponding to a count value of the photons) due to the influence of the pile-up, the count loss, or the like. In particular, in a case where the radiation dose is large, the linearity of the projection value with respect to the radiation dose deteriorates. The influence of the pile-up or the like varies with the energy bin. For example, in the bin having intermediate energy, the number of photons that are mixed in from a lower energy bin and the number of photons that overflow into a higher energy bin are similar, and thus the influence of the pile-up or the like is small. On the other hand, since the photons in the bin having lowest energy among the plurality of energy bins only overflow, the influence of the pile-up is large. Particularly, the linearity deteriorates in a region in which the projection value is low.

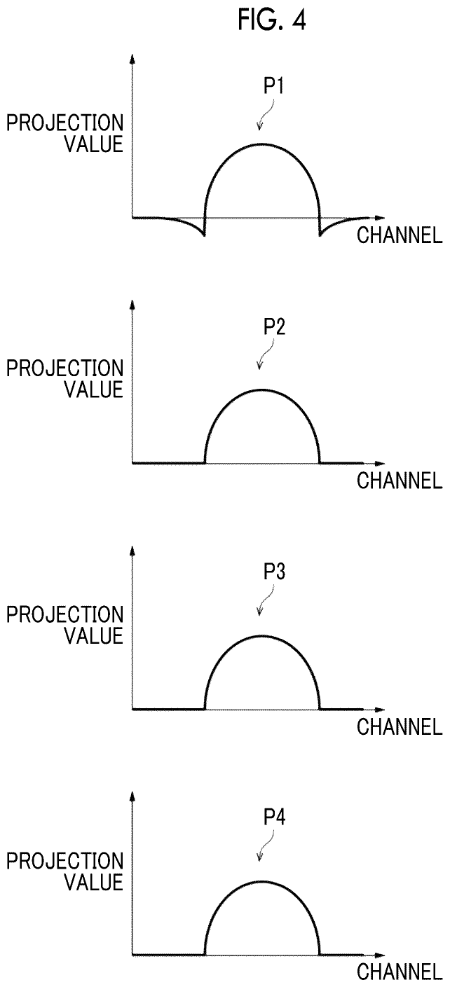

FIG. 4 is a diagram showing the deterioration of the linearity of the projection data for each of the energy bins. In FIG. 4, a horizontal axis represents a position in a channel direction in the detector 9, and a vertical axis represents a projection value. As shown in FIG. 4, in the first projection data P1 having the lowest energy among the four energy bins, the linearity of the projection value deteriorates in the region having a low projection value, and a negative projection value that is not actually possible appears. According to the experiment of the applicant, it has been confirmed that the third projection data P3 of the energy bin having the third lowest energy among the four energy bins has the highest linearity.

The first derivation unit 22 derives first correction data C1 for correcting the projection data of at least one target energy bin that is a correction target among the plurality of energy bins in order to correct the influence of the pile-up or the like. Specifically, the first derivation unit 22 derives the first correction data C1 by performing a weighted-combination of the projection values in the corresponding channels of the first to fourth projection data P1 to P4, as shown in Equation (1). In Equation (1), w1 to w4 are weighting coefficients for the first to fourth projection data P1 to P4.

C1=w1×P1+w2×P2+w3×P3+w4×P4 (1)

As described above, according to the experiment of the applicant, it has been confirmed that the third projection data P3 has the highest linearity. Therefore, the first derivation unit 22 sets the weighting coefficients w1 to w4 such that the weighting coefficient w3 among the weighting coefficients w1 to w4 is relatively larger than the other weighting coefficients. In the present embodiment, for example, the first correction data C1 may be derived by setting (w1, w2, w3, w4)=(0, 0, 1, 0). In such a case, the first correction data C1 matches the third projection data P3.

The first correction unit 23 derives the first corrected projection data H1 by correcting, in the projection data of the target energy bin, the projection value that is equal to or less than a first threshold value in the first correction data C1 using the first correction data C1. A region in which the first correction data C1 is equal to or less than the first threshold value corresponds to, for example, a region in which the projection value is low in the first projection data P1. In the present embodiment, the target energy bins are the first energy bin, the second energy bin, and the fourth energy bin, but the present disclosure is not limited to this. Only the first energy bin having the lowest linearity of the projection values may be used as the target energy bin.

For example, in a case of correcting the first projection data P1, the first correction unit 23 does not correct, in the first projection data P1, the projection value equal to or greater than a first threshold value Th1 in the first correction data C1, and sets the projection value of the first projection data P1 as a projection value of the first corrected projection data H1. In addition, the first correction unit 23 corrects the projection value that is equal to or greater than a second threshold value Th2, which is less than the first threshold value Th1, and less than the first threshold value Th1 in the first correction data C1 to the projection value derived by the interpolation operation between the projection value of the first projection data P1 and the projection value of the first correction data C1. Furthermore, the first correction unit 23 corrects the projection value less than the second threshold value Th2 in the first correction data C1 to the projection value of the first correction data C1.

As a result, the first correction unit 23 derives the first corrected projection data H1. For the second projection data P2 of the second energy bin, the third projection data P3 of the third energy bin, and the fourth projection data P4 of the fourth energy bin, the first corrected projection data H1 is also derived based on the first threshold value Th1 and the second threshold value Th2 in the first correction data C1, as in the first projection data P1. In the present embodiment, since the weighting coefficients in Equation (1) for deriving the first correction data C1 are set to (w1, w2, w3, w4)=(0, 0, 1, 0), the third projection data P3 is not corrected.

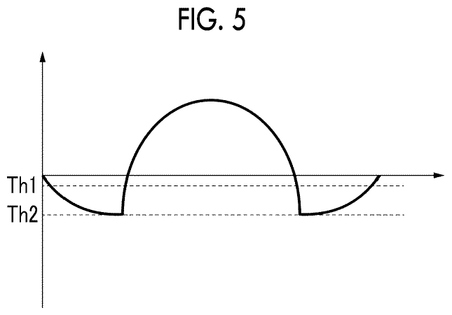

In a case where the first corrected projection data H1 is derived, the first correction unit 23 sets the first threshold value Th1 and the second threshold value Th2 for the first correction data C1. FIG. 5 is a diagram showing the settings of the first threshold value and the second threshold value. In the present embodiment, the bowtie filter 7 is used in the CT apparatus 2. As described above, the bowtie filter 7 increases the dose near the center and decreases the dose in the periphery to optimize the amount of exposure in order to suppress the amount of exposure in the peripheral portion. Here, the influence of the pile-up or the like increases as the dose increases. In FIG. 5, the first threshold value Th1 and the second threshold value Th2 are set after correcting the value of the first correction data C1 in accordance with the characteristics of the bowtie filter 7. As a result, in FIG. 5, the first threshold value Th1 and the second threshold value Th2 are set for the first correction data C1 such that the correction is performed from a lower value as the channel is closer to the center of the detector 9.

The first threshold value Th1 and the second threshold value Th2 may be set for each of the energy bins of the projection data that is the correction target. For example, the first threshold value Th1 and the second threshold value Th2 may be set to be smaller in a case of correcting the projection data of the energy bins that are less likely to be affected by the pile-up or the like than in a case of correcting the projection data of the energy bins that are likely to be affected by the pile-up or the like.

In addition, the first threshold value Th1 and the second threshold value Th2 may be set in accordance with the tube current in a case where the subject S is imaged. Here, since the dose of the radiation with which the subject S is irradiated is small in a case where the tube current is small, the influence of the pile-up or the like is small. Therefore, the first threshold value Th1 and the second threshold value Th2 may be relatively smaller as the tube current is smaller. The first threshold value Th1 and the second threshold value Th2 may be set in accordance with all of the characteristics of the bowtie filter 7, the energy bin, and the tube current, or the first threshold value Th1 and the second threshold value Th2 may be set in accordance with two of these values. Further, only the first threshold value Th1 may be set.

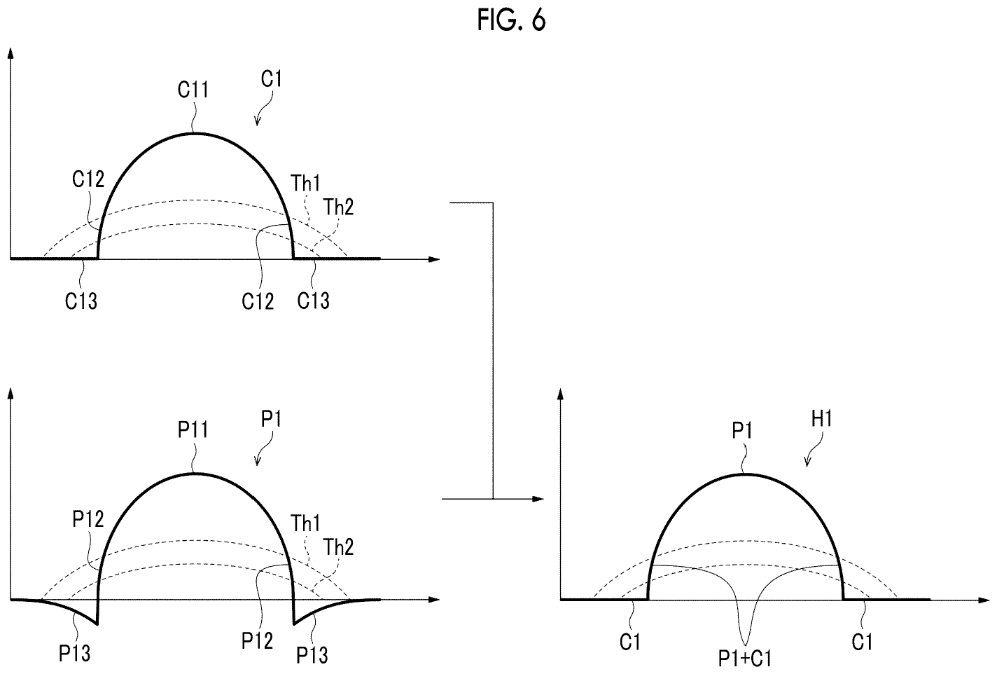

FIG. 6 is a diagram showing the derivation of the first corrected projection data. In FIG. 6, the derivation of the first corrected projection data H1 for the first projection data P1 will be described. In FIG. 6, unlike FIG. 5, the first correction data C1, the first projection data P1, and the first corrected projection data H1 are not corrected in consideration of the bowtie filter 7, so that the threshold values Th1 and Th2 are indicated by a curve. In FIG. 6, the first correction data C1 is divided into a data region C11 equal to or greater than the first threshold value Th1, a data region C12 equal to or greater than the second threshold value Th2 and less than the first threshold value Th1, and a data region C13 less than the second threshold value Th2. The first projection data P1 is divided into a data region P11 equal to or greater than the first threshold value Th1, a data region P12 equal to or greater than the second threshold value Th2 and less than the first threshold value Th1, and a data region P13 less than the second threshold value Th2 in the first correction data C1.

In the data region P11 of the first projection data P1, the first correction unit 23 sets the projection value of the first projection data P1 as it is as the projection value of the first corrected projection data H1. In the data region P12 of the first projection data P1, the projection value of the first corrected projection data H1 is derived by performing the interpolation operation between the projection value of the first projection data P1 and the projection value of the first correction data C1. In FIG. 6, the interpolation operation is represented by P1+C1. In the data region P13 of the first projection data P1, the first correction unit 23 sets the projection value of the first correction data C1 as it is as the projection value of the first corrected projection data H1. In this manner, the first correction unit 23 derives the first corrected projection data H1.

The interpolation operation in the data region P12 is performed by performing a weighted-addition of the projection value of the first correction data C1 and the projection value of the first projection data P1 in accordance with a difference between the projection value of the first correction data C1 and the first threshold value Th1 and the second threshold value Th2. Specifically, in a case where the projection value of the first correction data C1 is close to the first threshold value Th1, the first correction unit 23 assigns a smaller weight to the projection value of the first correction data C1 than to the projection value of the first projection data P1. On the contrary, in a case where the projection value of the first correction data C1 is close to the second threshold value Th2, a larger weight is assigned to the projection value of the first correction data C1 than to the projection value of the first projection data P1.

The second derivation unit 24 derives second correction data C2 for correcting the contrast of the first corrected projection data H1 in accordance with the size of the subject S based on the projection data in each of the plurality of energy bins. In the present embodiment, the second derivation unit 24 derives the second correction data C2 by performing a weighted-combination of the projection values in the corresponding channels of the first to fourth projection data P1 to P4 as shown in Equation (2). In Equation (2), w11 to w14 are weighting coefficients for the first to fourth projection data P1 to P4. The weighting coefficients w11 to w14 can be set to weights different from the weighting coefficients w1 to w4 in a case where the first derivation unit 22 derives the first correction data C1.

Here, it is more appropriate to use data having high linearity as an indicator of the size of the subject S. Therefore, the second derivation unit 24 derives the second correction data C2 by making the weighting coefficient w13 to the third projection data P3 having the highest linearity relatively larger than the weighting coefficients w11, w12, and w14 to the first projection data P1, the second projection data P2, and the fourth projection data P4, as described above.

C2=w11×P1+w12×P2+w13×P3+w14×P4 (2)

In the present embodiment, the second correction data C2 is derived by setting (w11, w12, w13, w14)=(0, 0, 1, 0). In such a case, the second correction data C2 matches the third projection data P3.

The second correction unit 25 sets the weight to the second correction data C2 and the weighting coefficient w20 to the first corrected projection data H1 in accordance with the size of the subject S. Then, the second correction unit 25 derives second corrected projection data H2 by performing a weighted-combination of the second correction data C2 and the first corrected projection data H1 using the set weighting coefficient w20.

The weighting coefficient w20 is set based on a predetermined relationship between the size of the subject S and the weighting coefficient w20. The relationship between the size of the subject S and the weighting coefficient w20 is derived in advance for each of the plurality of energy bins, and then stored in the storage 13.

In the present embodiment, the second correction data C2 matches the third projection data P3, and thus the second correction unit 25 corrects the contrasts of the first projection data P1, the second projection data P2, and the fourth projection data P4 (all of which are the first corrected projection data H1) with reference to the contrast of the third projection data P3. The first corrected projection data H1 for the first projection data P1, the second projection data P2, and the fourth projection data P4 is referred to as first corrected projection data H11, first corrected projection data H12, and first corrected projection data H14, respectively.

Here, the contrast of the projection data is increased or decreased in accordance with the size of the subject S. A change in the contrast of the projection data also varies with the energy bin. Therefore, in the present embodiment, the weighting coefficient is derived by measuring the change in the contrast in accordance with the size of the subject S in advance for each of the energy bins.

In the present embodiment, the contrasts of the first projection data P1, the second projection data P2, the third projection data P3, and the fourth projection data P4 in accordance with the size of the subject S are measured in advance by imaging the phantom. Here, in a case where a human body is imaged by radiography, a soft tissue having a composition close to water and a bone tissue containing a large amount of calcium are described as a typical example in which the contrast is high between substances constituting the human body. In water and calcium, the contrast is higher as the energy is lower, and the contrast is lower as the energy is higher. Therefore, for example, the phantom containing water and calcium is used, and the contrast in accordance with the size of the subject S is measured.

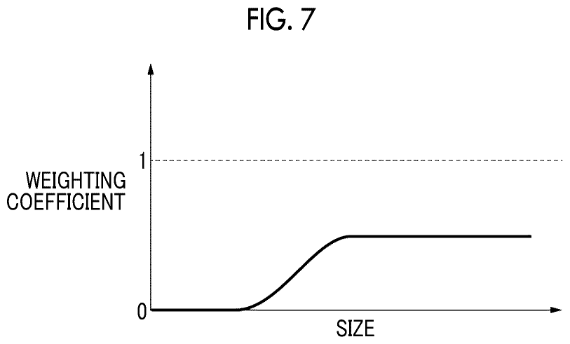

In the present embodiment, the relationship between the size of the subject S and the weighting coefficient w20 is derived in accordance with the measured contrast. For example, it is assumed that the contrast is higher as the subject S is larger, for the first projection data P1 (first corrected projection data H11). Therefore, in the present embodiment, the contrast of the first projection data P1 is corrected to be equivalent to the contrast in a case where the subject S has a small size. In such a case, for the first projection data P1, as shown in FIG. 7, the relationship between the size of the subject S and the weighting coefficient w20 is derived such that the weighting coefficient w20 is larger as the subject S is larger. The weighting coefficient w20 takes a value equal to or greater than 0 and equal to or less than a median value between 0 and 1. The second correction unit 25 derives the second corrected projection data H2 by performing a weighted-combination of the first corrected projection data H1 and the second correction data C2 by Equation (3). In Equation (3), the contrast of the first corrected projection data H1 is changed such that the contrast of the second corrected projection data H2 is closer to the contrast of the second correction data C2 as the weighting coefficient w20 is larger. In such a case, the contrast of the first corrected projection data H11 is reduced to be close to the second correction data C2.

H2=w20×C2+(1−w20)×H1 (3)

In the present embodiment, the second correction unit 25 adds the projection values of all the channels of the second correction data C2 and derives the square root of the added value as the indicator indicating the size of the subject S.

Meanwhile, in a case where the contrast of the first corrected projection data H11 is corrected to be small, the contrast of the second corrected projection data H2 of the first corrected projection data H11 is close to the contrast of the second projection data P2 (first corrected projection data H12). Therefore, for the first corrected projection data H12, the relationship between the subject S and the weighting coefficient w20 may be derived such that the contrast is close to the contrast of the second correction data C2 regardless of the size of the subject S. In such a case, the contrast of the first corrected projection data H12 is reduced to be close to the second correction data C2 by Equation (3).

In addition, for the contrast of the first corrected projection data H14, the weighting coefficient w20 in accordance with the size of the subject S may be derived in advance in accordance with the measured contrast, and the second corrected projection data H14 may be derived in accordance with Equation (3).

The reconstruction unit 26 acquires the second corrected projection data H2 at the plurality of projection angles from the storage 13 and performs the reconstruction process or the like to derive the tomographic image for each of the energy bins.

The display controller 27 displays the tomographic image derived by the reconstruction unit 26 on the display 14.

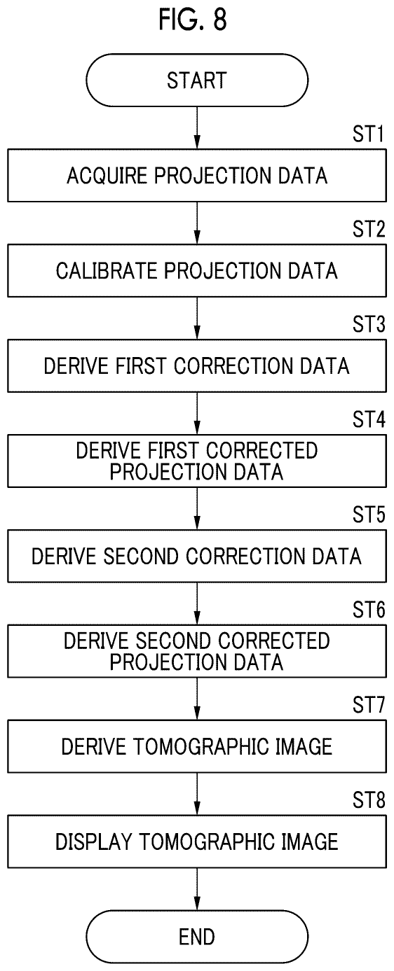

Hereinafter, a process performed in the present embodiment will be described. FIG. 8 is a flowchart showing the process performed in the present embodiment. In the CT apparatus 2, the subject S is imaged, and the information acquisition unit 20 acquires the projection data for each of the energy bins (step ST1). Then, the calibration unit 21 calibrates the projection data (step ST2), and the first derivation unit 22 derives the first correction data C1 (step ST3). Next, the first correction unit 23 derives the first corrected projection data H1 by correcting the projection data of the target energy bin using the first correction data C1 (step ST4).

Then, the second derivation unit 24 derives the second correction data C2 for adjusting the contrast (step ST5), and the second correction unit 25 derives the second corrected projection data H2 of the target energy bin by correcting the first corrected projection data H1 using the second correction data C2 (step ST6). Then, the reconstruction unit 26 derives the tomographic image by reconstructing the second corrected projection data H2 (Step ST7), the display controller 27 displays the tomographic image on the display 14 (Step ST8), and the process ends.

Here, the influence of the pile-up or the like appears in a region in which the projection value is relatively low in the projection data. In the present embodiment, the first correction data C1 for correcting the projection value equal to or less than the first threshold value included in the projection data of at least one target energy bin that is the correction target among the plurality of energy bins is derived based on the projection data in each of the plurality of energy bins, and the projection value equal to or less than the first threshold value included in the projection data of the target energy bin is corrected based on the first correction data C1. Therefore, it is possible to appropriately correct the influence of the pile-up or the like.

In addition, in the present embodiment, the contrast of the first corrected projection data H1 is corrected in accordance with the size of the subject S. Therefore, it is possible to suppress the variation in the contrast of the projection data for each of the energy bins in accordance with the size of the subject S, and as a result, it is possible to derive the tomographic image with a higher image quality.

In the embodiment described above, the second derivation unit 24 and the second correction unit 25 are provided, and the contrast of the first corrected projection data H1 is corrected, but the present disclosure is not limited to this. Only the first corrected projection data H1 may be derived with no correction of the contrast. In such a case, the reconstruction unit 26 need only derive the tomographic image by reconstructing the first corrected projection data H1.

In addition, in the embodiment described above, the photon-counting detector outputs detection signals of four energy bands, but the present disclosure is not limited to this. Raw data of a plurality of energy bands less than four or four or more may be output.

In addition, in the embodiment described above, the processor includes, in addition to the CPU which is a general-purpose processor that executes software (program) to function as various processing units, a programmable logic device (PLD) such as a field-programmable gate array (FPGA) whose circuit configuration can be changed after manufacture, and a dedicated electric circuit which is a processor having a circuit configuration dedicatedly designed to execute specific processing such as an application-specific integrated circuit (ASIC).

Further, the various processes described above may be executed by one of the various processors, or may be executed by a combination of two or more processors (for example, a combination of a plurality of FPGAs or a CPU and an FPGA) of the same type or different types. In addition, a plurality of processing units may be configured by one processor. Examples in which the plurality of processing units are configured by one processor include a form in which a processor that implements all functions of a system including the plurality of processing units by using one integrated circuit (IC) chip is used, such as a system on a chip (SOC).

The supplementary notes of the present disclosure will be described as follows.

### Supplementary Note 1

A data processing device comprising: a processor configured to acquire projection data that is obtained by detecting radiation emitted from a radiation source and transmitted through a subject via a photon-counting detector consisting of a plurality of detection elements and that corresponds to the number of photons of the radiation in each of a plurality of energy bins of the radiation, derive first correction data for correcting projection data of at least one target energy bin that is a correction target among the plurality of energy bins based on the projection data in each of the plurality of energy bins, and derive first corrected projection data by correcting, in the projection data of the target energy bin, a projection value that is equal to or less than a first threshold value in the first correction data using the first correction data.

### Supplementary Note 2

The data processing device according to supplementary note 1, in which the processor is configured to calibrate the projection data in each of the plurality of energy bins, and derive the first correction data and the first corrected projection data using the calibrated projection data.

### Supplementary Note 3

The data processing device according to supplementary note 1 or 2, in which the processor is configured to derive the first corrected projection data by correcting, in the projection data of the target energy bin, a projection value that is equal to or greater than a second threshold value, which is less than the first threshold value, and less than the first threshold value in the first correction data to a projection value derived by an interpolation operation between the projection value of the projection data of the target energy bin and the projection value of the first correction data, and correcting a projection value that is less than the second threshold value in the first correction data to the projection value of the first correction data.

### Supplementary Note 4

The data processing device according to supplementary note 3, in which the processor is configured to set at least one of the first threshold value or the second threshold value based on at least one of energy of the target energy bin, positions of the detection elements in accordance with a shape of a compensator that compensates a dose of the radiation emitted to the subject, or a tube current of the radiation.

### Supplementary Note 5

The data processing device according to any one of supplementary notes 1 to 4, in which the target energy bin is at least one of a maximum energy bin or a minimum energy bin among the plurality of energy bins, and the processor is configured to derive the first correction data by assigning a relatively larger weight to the projection data of energy bins other than the maximum energy bin and the minimum energy bin among the plurality of energy bins than to the projection data of the maximum energy bin and the minimum energy bin, and performing a weighted-combination of a plurality of the projection data.

### Supplementary Note 6

The data processing device according to any one of supplementary notes 1 to 5, in which the processor is configured to derive second correction data for correcting a contrast of the first corrected projection data in accordance with a size of the subject based on the projection data in each of the plurality of energy bins, and derive second corrected projection data by performing a weighted-combination of the second correction data and the first corrected projection data in accordance with the size of the subject.

### Supplementary Note 7

The data processing device according to supplementary note 6, in which the processor is configured to derive the second correction data by assigning a relatively larger weight to the projection data of an energy bin in which a variation in the projection value due to the size of the subject is relatively smaller than other energy bins among the plurality of energy bins than to the projection data of the other energy bins, and performing a weighted-combination of a plurality of the projection data.

### Supplementary Note 8

The data processing device according to supplementary note 6 or 7, in which the processor is configured to derive an indicator indicating the size of the subject based on an added value of the projection values of the second correction data.

### Supplementary Note 9

A data processing method executed by a computer, comprising: acquiring projection data that is obtained by detecting radiation emitted from a radiation source and transmitted through a subject via a photon-counting detector consisting of a plurality of detection elements and that corresponds to the number of photons of the radiation in each of a plurality of energy bins of the radiation; deriving first correction data for correcting projection data of at least one target energy bin that is a correction target among the plurality of energy bins based on the projection data in each of the plurality of energy bins; and deriving first corrected projection data by correcting a projection value, which is equal to or less than a first threshold value in the first correction data, in the projection data of the target energy bin by the first correction data.

### Supplementary Note 10

A data processing program causing a computer to execute a procedure comprising: acquiring projection data that is obtained by detecting radiation emitted from a radiation source and transmitted through a subject via a photon-counting detector consisting of a plurality of detection elements and that corresponds to the number of photons of the radiation in each of a plurality of energy bins of the radiation; deriving first correction data for correcting projection data of at least one target energy bin that is a correction target among the plurality of energy bins based on the projection data in each of the plurality of energy bins; and deriving first corrected projection data by correcting a projection value, which is equal to or less than a first threshold value in the first correction data, in the projection data of the target energy bin by the first correction data.

What is claimed is:

1. A data processing device comprising:
a processor configured to
acquire projection data that is obtained by detecting radiation emitted from a radiation source and transmitted through a subject via a photon-counting detector consisting of a plurality of detection elements and that corresponds to the number of photons of the radiation in each of a plurality of energy bins of the radiation,
derive first correction data for correcting projection data of at least one target energy bin that is a correction target among the plurality of energy bins based on the projection data in each of the plurality of energy bins, and
derive first corrected projection data by correcting, in the projection data of the target energy bin, a projection value that is equal to or less than a first threshold value in the first correction data using the first correction data.

2. The data processing device according to claim 1,
wherein the processor is configured to
calibrate the projection data in each of the plurality of energy bins, and
derive the first correction data and the first corrected projection data using the calibrated projection data.

3. The data processing device according to claim 1,
wherein the processor is configured to derive the first corrected projection data by correcting, in the projection data of the target energy bin, a projection value that is equal to or greater than a second threshold value, which is less than the first threshold value, and less than the first threshold value in the first correction data to a projection value derived by an interpolation operation between the projection value of the projection data of the target energy bin and the projection value of the first correction data, and correcting a projection value that is less than the second threshold value in the first correction data to the projection value of the first correction data.

4. The data processing device according to claim 3,
wherein the processor is configured to set at least one of the first threshold value or the second threshold value based on at least one of energy of the target energy bin, positions of the detection elements in accordance with a shape of a compensator that compensates a dose of the radiation emitted to the subject, or a tube current of the radiation.

5. The data processing device according to claim 1,
wherein the target energy bin is at least one of a maximum energy bin or a minimum energy bin among the plurality of energy bins, and
the processor is configured to derive the first correction data by assigning a relatively larger weight to the projection data of energy bins other than the maximum energy bin and the minimum energy bin among the plurality of energy bins than to the projection data of the maximum energy bin and the minimum energy bin, and performing a weighted-combination of a plurality of the projection data.

6. The data processing device according to claim 1,
wherein the processor is configured to
derive second correction data for correcting a contrast of the first corrected projection data in accordance with a size of the subject based on the projection data in each of the plurality of energy bins, and
derive second corrected projection data by performing a weighted-combination of the second correction data and the first corrected projection data in accordance with the size of the subject.

7. The data processing device according to claim 6,
wherein the processor is configured to derive the second correction data by assigning a relatively larger weight to the projection data of an energy bin in which a variation in the projection value due to the size of the subject is relatively smaller than other energy bins among the plurality of energy bins than to the projection data of the other energy bins, and performing a weighted-combination of a plurality of the projection data.

8. The data processing device according to claim 6,
wherein the processor is configured to derive an indicator indicating the size of the subject based on an added value of the projection values of the second correction data.

9. A data processing method executed by a computer, comprising:
acquiring projection data that is obtained by detecting radiation emitted from a radiation source and transmitted through a subject via a photon-counting detector consisting of a plurality of detection elements and that corresponds to the number of photons of the radiation in each of a plurality of energy bins of the radiation;
deriving first correction data for correcting projection data of at least one target energy bin that is a correction target among the plurality of energy bins based on the projection data in each of the plurality of energy bins; and
deriving first corrected projection data by correcting, in the projection data of the target energy bin, a projection value that is equal to or less than a first threshold value in the first correction data using the first correction data.

10. A non-transitory computer-readable storage medium that stores a data processing program causing a computer to execute a procedure comprising:
acquiring projection data that is obtained by detecting radiation emitted from a radiation source and transmitted through a subject via a photon-counting detector consisting of a plurality of detection elements and that corresponds to the number of photons of the radiation in each of a plurality of energy bins of the radiation;
deriving first correction data for correcting projection data of at least one target energy bin that is a correction target among the plurality of energy bins based on the projection data in each of the plurality of energy bins; and
deriving first corrected projection data by correcting, in the projection data of the target energy bin, a projection value that is equal to or less than a first threshold value in the first correction data using the first correction data.
